(19) United States
(12) Patent Application Publication
(10) Pub. No.: US 2009/0221924 A1
(43) Pub. Date: Sep. 3, 2009

(54) METHOD AND SYSTEM FOR NON-INVASIVE BLOOD PRESSURE DETERMINATION

(75) Inventors: Bruce Arnold Friedman, Tampa, FL (US); Richard Medero, Tampa, FL (US); Lawrence T. Hersh, Tampa, FL (US); Sai Kolluri, Tampa, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/040,379

(22) Filed: Feb. 29, 2008

(51) Int. Cl. A61B 5/022

(52) U.S. Cl. 600/490

(57) ABSTRACT

A method for non-invasively estimating blood pressure is disclosed herein. The method includes inflating a cuff and collecting first oscillation amplitude data at a first plurality of cuff pressure levels while inflating the cuff. The method also includes deflating the cuff and collecting second oscillation amplitude data at a second plurality of cuff pressure levels while deflating the cuff. The method also includes fitting a curve to the first oscillation amplitude data and to the second oscillation amplitude data and estimating a blood pressure parameter based on the curve. A non-invasive blood pressure system is also disclosed.

### BACKGROUND OF THE INVENTION

The subject matter disclosed herein relates to a method and system for non-invasive blood pressure determination.

Human heart muscles periodically contract, forcing blood through the arteries. As a result of this pumping action, pressure pulses exist in these arteries and cause them to cyclically change volume. The minimum pressure for these pulses during a cardiac cycle is known as the diastolic pressure and the peak pressure is known as the systolic pressure. A further pressure parameter, known as the “mean arterial pressure” (MAP), represents a time-weighted average of the blood pressure. Blood pressure parameters such as systolic pressure, MAP and diastolic pressure for a patient are useful in monitoring the cardiovascular state of the patient, and in treating disease.

A conventional method of measuring blood pressure is referred to as oscillometry. Typically, the measurement of blood pressure by oscillometry requires the inflation of a cuff to a cuff pressure level above the patient's systolic pressure to fully occlude the artery. Blood pressure is then determined by measuring an oscillation amplitude value at multiple cuff pressure levels during the deflation of the cuff. The patient's systolic pressure is not known during the initial inflation. One problem with the conventional method is that the cuff may be inflated to an unnecessarily high cuff pressure level since the patient's systolic pressure is not known during the initial inflation of the cuff. This may lead to patient discomfort. Another problem with the conventional method is that if the initial cuff pressure level is too low, it may be necessary to inflate the cuff to a higher pressure level as part of one or more subsequent steps. If subsequent steps are required to reach a correct initial cuff pressure level, this causes the blood pressure measurement to take longer than necessary. Additionally, the extra time needed to inflate the cuff to the right cuff pressure level may also lead to patient discomfort.

### BRIEF DESCRIPTION OF THE INVENTION

The above-mentioned shortcomings, disadvantages and problems are addressed herein which will be understood by reading and understanding the following specification.

In an embodiment, a method for non-invasively estimating blood pressure includes inflating a cuff and collecting first oscillation amplitude data at a first plurality of cuff pressure levels while inflating the cuff. The method also includes deflating the cuff and collecting second oscillation amplitude data at a second plurality of cuff pressure levels while deflating the cuff. The method also includes fitting a curve to the first oscillation amplitude data and to the second oscillation amplitude data and estimating a blood pressure parameter based on the curve.

In another embodiment, a method for non-invasively estimating blood pressure includes inflating a cuff and collecting first oscillation amplitude data at a first plurality of cuff pressure levels while inflating the cuff. The method also includes deflating the cuff and collecting second oscillation amplitude data at a second plurality of cuff pressure levels while deflating the cuff. At least one of the second plurality of cuff pressure levels differs from each of the first plurality of cuff pressure levels. The method also includes fitting a curve to the first oscillation amplitude data and to the second oscillation amplitude data and estimating a blood pressure parameter based on the curve.

In another embodiment, a system for non-invasively estimating a blood pressure parameter includes a cuff, a source of pressurized gas attached to the cuff, a deflation valve attached to the cuff, and a transducer attached to the cuff. The transducer is configured to acquire a first plurality of oscillation amplitude values while the cuff is inflated by the source of pressurized gas and the transducer is further configured to acquire a second plurality of oscillation amplitude values while the cuff is deflated via the deflation valve. The system also includes a controller attached to the cuff, the transducer, the source of pressurized gas, and the deflation valve. The controller is configured to fit a curve to the first plurality of oscillation amplitude values and to the second plurality of oscillation amplitude values and estimate a blood pressure parameter based on the curve.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a non-invasive blood pressure system in accordance with an embodiment;

FIG. 2 is a flow chart illustrating a method in accordance with an embodiment;

FIG. 3 is a cuff pressure level versus time plot in accordance with an embodiment; and

FIG. 4 is graphical representation of oscillation amplitude versus cuff pressure level in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

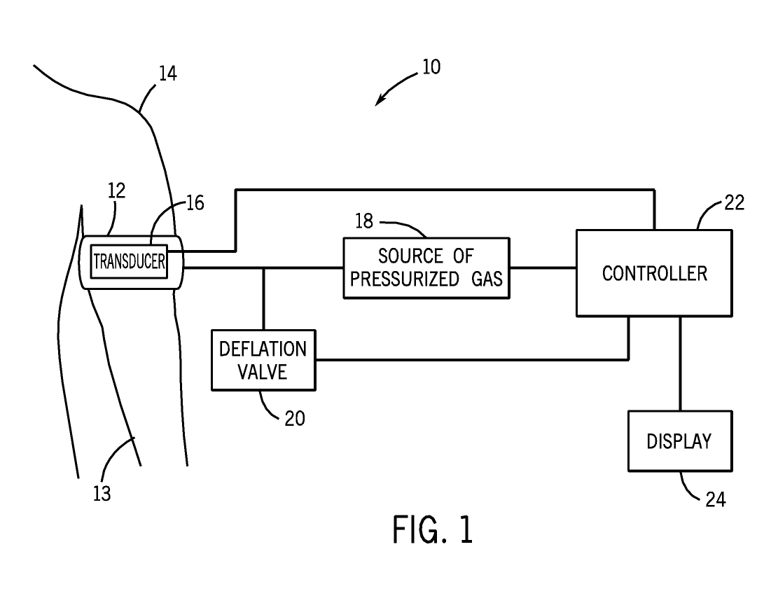

Referring to FIG. 1, a schematic representation of a non-invasive blood pressure (NIBP) system 10 is shown in accordance with an embodiment. The NIBP system 10 includes a cuff 12 disposed about an arm 13 of a patient 14. The cuff 12 comprises one or more inflatable bladders (not shown) that can be selectively filled with air. While the cuff 12 is depicted around the arm 13 of the patient 14 in this embodiment, it should be appreciated that the cuff 12 could also be disposed about a leg or any other limb.

A transducer 16 is attached to the cuff 12 and configured to obtain a cuff pressure signal. The cuff pressure signal is used to determine measurements of a cuff pressure level and an oscillation amplitude. For the purposes of this disclosure, the “cuff pressure level” is defined to include a lower frequency portion of the cuff pressure signal, while the “oscillation amplitude” is defined to include the amplitude of a higher frequency portion of the cuff pressure signal that varies with the expansion and contraction of the patient's arteries. Both cuff pressure level and oscillation amplitude are well-known values in the oscillometric field.

A source of pressurized gas 18 is connected to the cuff 12 in a manner that allows gas to travel into the cuff 12 to increase the cuff pressure level. A deflation valve 20 is also connected to the cuff 12 and the deflation valve 20 functions to selectively lower the cuff pressure level. A controller 22 is operatively connected to the transducer 16, the source of pressurized gas 18, the deflation valve 20, and a display 24. The controller 22 is configured to regulate the source of pressurized gas 18 and the deflation valve 20 in order to attain a desired cuff pressure level. The display 24 is attached to the controller 20 and is adapted to display a blood pressure parameter as will be discussed in detail hereinafter.

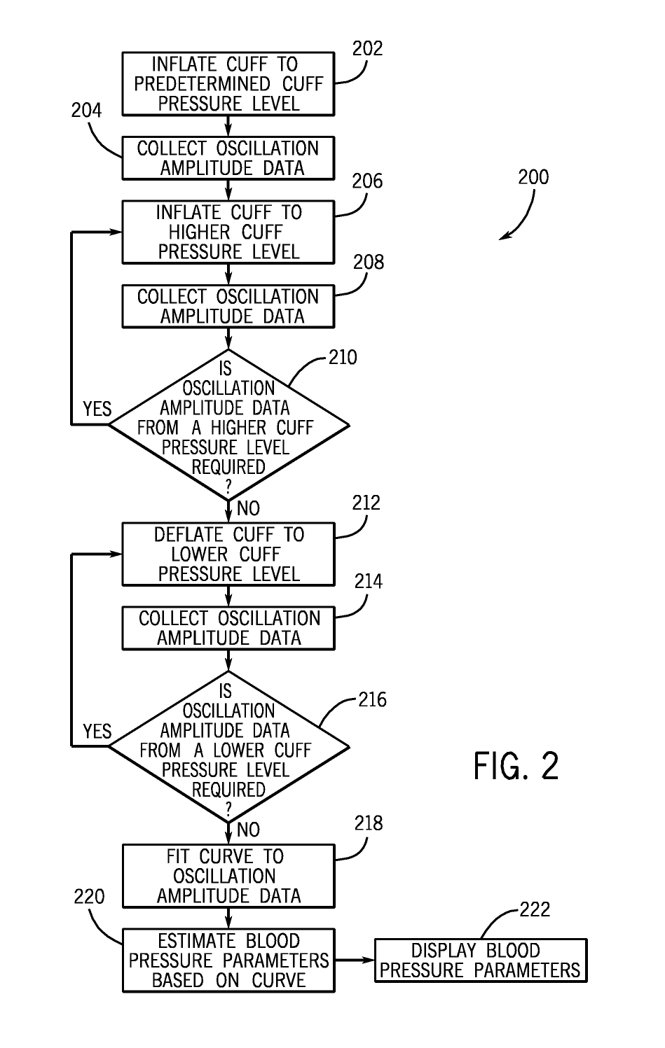

FIG. 2 is a flow chart illustrating a method 200 in accordance with an embodiment. The individual blocks 202-222 of the flow chart represent steps that may be performed in accordance with the method 200. The technical effect of method 200 is the estimation of blood pressure parameters based on oscillation amplitude data collected both during an inflation process of the cuff 12 (shown in FIG. 1) and during a deflation process of the cuff 12. Steps 202-222 of the method 200 need not be performed in the order shown.

Referring to FIGS. 1 and 2, at step 202, the cuff 12 is inflated to a predetermined cuff pressure level. The controller 22 controls the source of pressurized gas 18 and the deflation valve 20 in a manner adapted to bring the cuff pressure level close to the predetermined cuff pressure level. The predetermined cuff pressure level may be based on data from a previous estimation of the patient's blood pressure, it may be based on empirical data, or it may be manually set by an operator.

At step 204, oscillation amplitude data is collected at the predetermined cuff pressure level of step 202. For the purposes of this disclosure, it should be understood that oscillation amplitude data may comprise an oscillation amplitude value or a plurality of oscillation amplitude values.

At step 206, the cuff 12 is inflated to a higher cuff pressure level. At step 208, oscillation amplitude data is collected at the higher cuff pressure level of step 206. At step 210, after the oscillation amplitude data has been collected, the controller 22 determines if oscillation amplitude data from a higher cuff pressure level is required. This determination may be made by comparing the number of cuff pressure levels from which oscillation amplitude data has been collected to a previously determined number of cuff pressure levels needed for a specific blood pressure estimation. It should be appreciated by those skilled in the art that additional methods of determining if oscillation amplitude data from a higher cuff pressure level is required may also be employed at step 210.

If oscillation amplitude data from a higher cuff pressure level is required at step 210, the method 200 returns back to step 206, where the cuff 12 is inflated to a higher cuff pressure level. As the method 200 iteratively cycles through steps 206 through 210, the cuff pressure level is increased in either a stepwise or a continuous manner. For the purposes of this disclosure, “increased in a stepwise manner” is defined to include an inflation process where the cuff pressure level is increased in steps and where the cuff pressure level is maintained at a generally constant value at times when the oscillation amplitude data is collected. For the purposes of this disclosure, “increased in a continuous manner” is defined to include an inflation process where the cuff pressure level is continuously increased while oscillation amplitude data is collected. It should be appreciated by those skilled in the art that “increased in a continuous manner” includes methods employing both a generally constant rate of inflation of the cuff 12 and a variable rate of inflation of the cuff 12. It should also be appreciated that it may be possible to adaptively change either the rate of inflation or the size of the steps between cuff pressure levels depending upon the oscillation amplitude data collected during the steps 202-210.

By collecting data while increasing the cuff pressure level according to steps 206-210, it is also possible to ensure that the cuff 12 is not inflated to an unnecessarily high cuff pressure level. For example, if the NIBP system 10 is no longer obtaining oscillation amplitude values because the patient's 14 artery is fully occluded, it may not be necessary to inflate the cuff 12 to a higher cuff pressure level.

If oscillation amplitude data from a higher cuff pressure level is not required at step 210, the method 200 proceeds to step 212. At step 212, the controller 22 controls the deflation valve 20 to deflate the cuff 12 to a lower cuff pressure level. At step 214, oscillation amplitude data is collected from the lower cuff pressure level. The collection of oscillation amplitude data at steps 204, 208, and 214 will be described in accordance with an illustrative embodiment wherein the collection of oscillation amplitude data comprises collecting a single oscillation amplitude value at each cuff pressure level. It should, however, be appreciated that in alternate embodiments, the collection of oscillation amplitude data at steps 204, 208, and 214 may comprise collecting multiple oscillation amplitude values at each cuff pressure level. The collection of multiple oscillation amplitude values at each cuff pressure level may be implemented to provide a type of quality check. For example, if multiple oscillation amplitude values are collected at a single cuff pressure level, a comparison between the multiple oscillation amplitude values may be conducted. If the multiple oscillation amplitude values collected at a single cuff pressure level are found to vary by more than a predetermined amount, this may be a sign that one or more of the oscillation amplitude values contains an artifact. According to an embodiment, it may be desirable to collect additional oscillation amplitude values at a specific cuff pressure level if the multiple oscillation amplitude values collected at a single cuff pressure level are found to vary by more than the predetermined amount. According to an embodiment, the controller 22 may control the deflation of the cuff 12 during step 212 in a manner so that the cuff pressure level where oscillation amplitude data is collected at step 214 is different from the cuff pressure levels where oscillation amplitude data was collected at steps 204 and 208.

At step 216, the controller 22 determines if oscillation amplitude data from a lower cuff pressure level is required. This determination may, for example, be made by calculating if oscillation amplitude data from enough cuff pressure levels has been collected at steps 204, 208, and 214 in order to complete an oscillometric envelope for a specific blood pressure estimation as is known by those skilled in the art. If oscillation amplitude data from an additional lower cuff pressure level is needed to complete the oscillometric envelope, then the method 200 returns back to step 212, where the cuff 12 is deflated to a lower cuff pressure level. It should be appreciated that additional methods of determining if oscillation amplitude data from a lower cuff pressure level is required may also be employed. As the method 200 iteratively cycles through steps 212 through 216, the cuff pressure level is decreased in either a stepwise manner or a continuous manner. For the purposes of this disclosure, “decreased in a stepwise manner” is defined to include a deflation process where the cuff pressure level is decreased in steps and where the cuff pressure level is maintained at a generally constant value at times when the oscillation amplitude data is collected. For the purposes of this disclosure, “decreased in a continuous manner” is defined to include a deflation process where the cuff pressure level is continuously decreased while oscillation amplitude data is collected. It should be appreciated by those skilled in the art that “decreased in a continuous manner” includes methods employing both a generally constant rate of deflation of the cuff 12 and a variable rate of deflation of the cuff 12. It should also be appreciated that it may be possible to adaptively change either the rate of deflation or the size of the steps between cuff pressure levels. It should be understood that it may also be possible to use additional patterns of inflation and deflation in order to most efficiently acquire oscillation amplitude data.

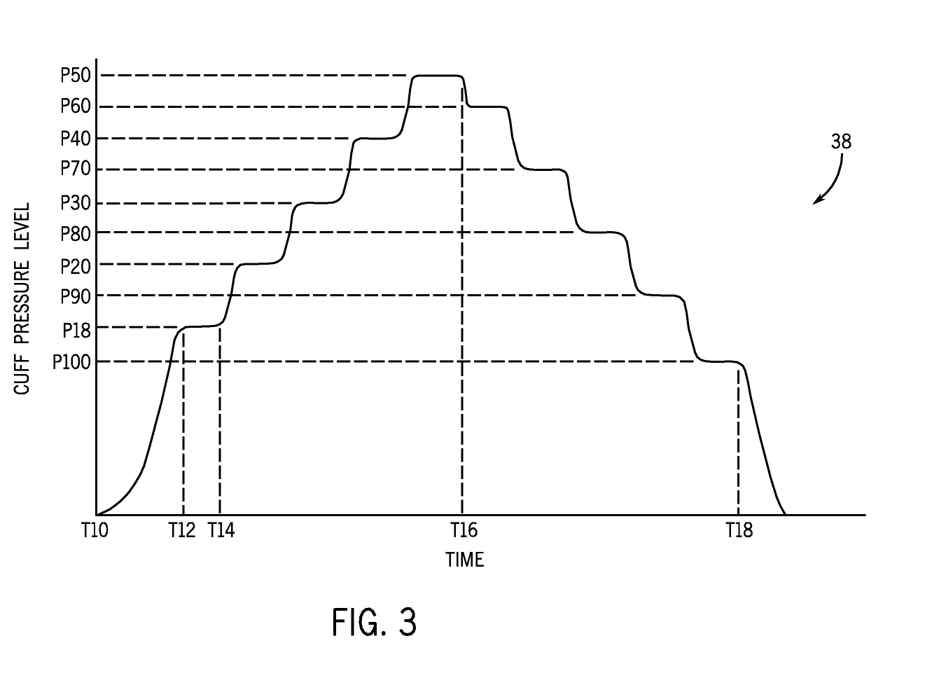

Referring to FIGS. 2 and 3, a cuff pressure level versus time plot 38 of steps 202-216 of an embodiment of the method 200 is shown. From a time T10 until a time T12, the cuff pressure level is increased to a predetermined cuff pressure level, graphically represented by cuff pressure level P18, according to step 202. According to an embodiment, the cuff pressure level is generally maintained at the cuff pressure level P18 for a period of time as indicated by the generally horizontal portion of the graph from the time T12 to a time T14. Between the time T12 and the time T14, oscillation amplitude data is collected according to step 204. From the time T14 until a time T16, the cuff pressure level is increased according to steps 206 through 210. According to an embodiment, the collection of oscillation amplitude data at step 208 takes place at times when the cuff pressure level is generally constant, such as at cuff pressure levels P20, P30, P40, and P50.

From the time T16 until a time T18, the cuff pressure level is decreased according to the steps 212-216. According to an embodiment, the collection of oscillation amplitude data at step 214 takes place at times when the cuff pressure level is generally constant. In the embodiment depicted in FIG. 3, the decreasing cuff pressure levels (i.e. the cuff pressure levels P60, P70, P80, P90, and P100) are all distinct from the increasing cuff pressure levels (i.e. the cuff pressure levels P18, P20, P30, P40, and P50). An example of this is depicted by the cuff pressure level P60, which is shown as being between the cuff pressure level P50 and the cuff pressure level P40. It should be understood that in additional embodiments some or all of the decreasing cuff pressure levels may be the same as the increasing cuff pressure levels.

After oscillation amplitude data is collected for the cuff pressure level P100, the controller 22 (shown in FIG. 1) determines that no additional deflation steps are required in accordance with an exemplary embodiment. Since additional oscillation amplitude data is not required, the controller 22 stops collecting data for the patient 14 (shown in FIG. 1). If oscillation amplitude data from a lower cuff pressure level is not required at step 216, the method 200 proceeds to step 218.

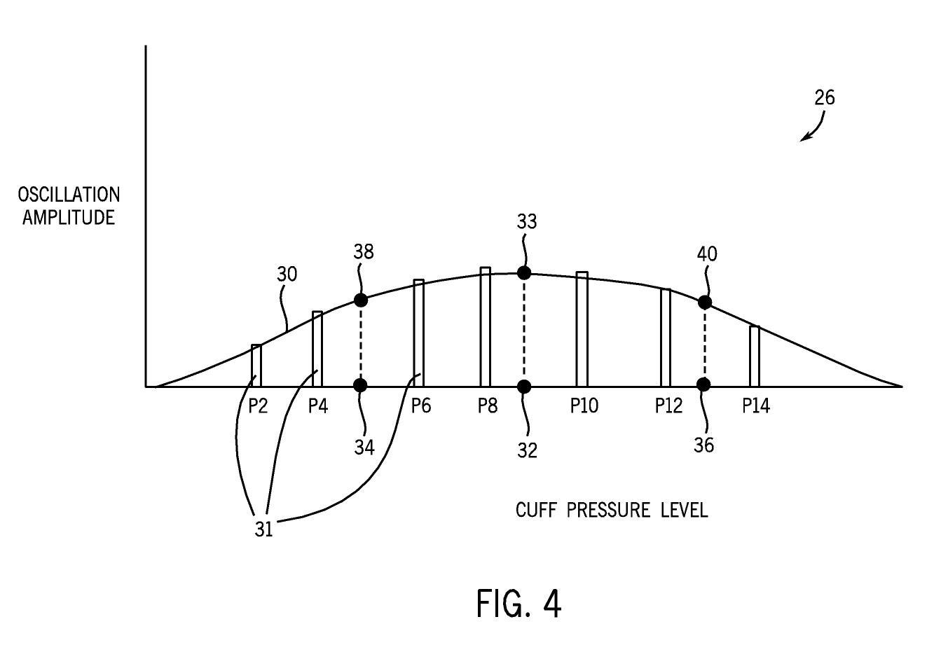

Referring to FIGS. 2 and 4, at step 218 a curve 30 is fit to the oscillation amplitude data collected at steps 204, 208, and 214. Fitting a curve is a well known mathematical technique and may comprise fitting one of the following nonlimiting list of functions to the oscillation amplitude data: linear, piecewise linear, quadratic, exponential, and Gaussian. Different functions used for the curve may be chosen depending upon the preferred properties of the blood pressure estimation and the amount of oscillation amplitude data available.

At step 220, one or more blood pressure parameters of the patient 14 (shown in FIG. 1) are estimated based on the curve 30. For this exemplary embodiment, the oscillation amplitude data comprises oscillation amplitude values 31 that are depicted as bars on a graphical representation 26 of oscillation amplitude versus cuff pressure level. The curve 30 is implemented to determine an estimate of a mean arterial pressure 32 of the patient 14. The mean arterial pressure 32 may be estimated by implementing the curve 30 to find the cuff pressure level where the curve 30 reaches a maximum oscillation amplitude 33. Once an estimate of the mean arterial pressure 32 has been made, estimates of a diastolic pressure 34 and a systolic pressure 36 may be made based on well-known relationships of the diastolic pressure 34 compared to the mean arterial pressure 32 and the systolic pressure 36 compared to the mean arterial pressure 32. According to an exemplary embodiment, the diastolic pressure 34 may be estimated by finding the cuff pressure level below the mean arterial pressure 32 where the ratio of the oscillation amplitude at the diastolic pressure 34 to the oscillation amplitude at the mean arterial pressure 32 equals a first established value, typically chosen to be between 0.4 and 1.0. A point 38 represents the location on the curve 30 where the ratio of the oscillation amplitude at the diastolic pressure 34 to the oscillation amplitude at the mean arterial pressure 32 equals a first established value. According to an exemplary embodiment, the systolic pressure 36 may be estimated by finding the cuff pressure level above the mean arterial pressure 32 where the ratio of the oscillation amplitude at the systolic pressure 36 to the oscillation amplitude at the mean arterial pressure 32 equals a second established value, typically chosen to be between 0.4 and 1.0. A point 40 represents the location on the curve 30 where the ratio of the oscillation amplitude at the systolic pressure 36 to the oscillation amplitude at the mean arterial pressure 32 equals a second established value. While the mean arterial pressure 32, the systolic pressure 36, and the diastolic pressure 34 are examples of blood pressure parameters, it should be understood that it would be possible to use the curve 30 to estimate other blood pressure parameters as well.

Referring to FIG. 2, at step 222, the blood pressure parameters estimated at step 220 are displayed. It is within the scope of this invention for the blood pressure parameters to be displayed as a numeral, a graph, or any other form that conveys the blood pressure parameters to an observer.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for non-invasively estimating blood pressure comprising:
inflating a cuff;
collecting first oscillation amplitude data at a first plurality of cuff pressure levels while said inflating the cuff;
deflating the cuff;
collecting second oscillation amplitude data at a second plurality of cuff pressure levels while said deflating the cuff;
fitting a curve to the first oscillation amplitude data and to the second oscillation amplitude data; and
estimating a blood pressure parameter based on the curve.

2. The method of claim 1, wherein said inflating the cuff comprises inflating the cuff in a stepwise manner.

3. The method of claim 2, wherein said deflating the cuff comprises deflating the cuff in a continuous manner.

4. The method of claim 2, wherein said deflating the cuff comprises deflating the cuff in a stepwise manner.

5. The method of claim 1, wherein said inflating the cuff comprises inflating the cuff in a continuous manner.

6. The method of claim 5, wherein said deflating the cuff comprises deflating the cuff in a stepwise manner.

7. The method of claim 5, wherein said deflating the cuff comprises deflating the cuff in a continuous manner.

8. The method of claim 1, further comprising analyzing the first oscillation amplitude data and the second oscillation amplitude data to determine if either the first oscillation amplitude data or the second oscillation amplitude data contains an artifact.

9. The method of claim 8, further comprising collecting additional oscillation amplitude data if the first oscillation amplitude data or the second oscillation amplitude data for the cuff pressure level contains an artifact.

10. A method for non-invasively estimating blood pressure comprising:
inflating a cuff;
collecting first oscillation amplitude data at a first plurality of cuff pressure levels while said inflating the cuff;
deflating the cuff;
collecting second oscillation amplitude data at a second plurality of cuff pressure levels while said deflating the cuff, at least one of said second plurality of cuff pressure levels differing from each of said first plurality of cuff pressure levels;
fitting a curve to the first oscillation amplitude data and to the second oscillation amplitude data; and
estimating a blood pressure parameter based on the curve.

11. The method of claim 10, wherein said inflating the cuff comprises inflating the cuff in a stepwise manner.

12. The method of claim 11, wherein said deflating the cuff comprises deflating the cuff in a continuous manner.

13. The method of claim 11, wherein said deflating the cuff comprises deflating the cuff in a stepwise manner.

14. The method of claim 10, where in said inflating the cuff comprises inflating the cuff in a continuous manner.

15. The method of claim 14, wherein said deflating the cuff comprises deflating the cuff in a continuous manner.

16. The method of claim 14, wherein said deflating the cuff comprises deflating the cuff in a stepwise manner.

17. A system for non-invasively estimating a blood pressure parameter comprising:
a cuff;
a source of pressurized gas attached to the cuff;
a deflation valve attached to the cuff;
a transducer attached to the cuff, wherein the transducer is configured to acquire a first plurality of oscillation amplitude values while the cuff is inflated by the source of pressurized gas and wherein the transducer is further configured to acquire a second plurality of oscillation amplitude values while the cuff is deflated via the deflation valve; and
a controller attached to the transducer, wherein said controller is configured to fit a curve to the first plurality of oscillation amplitude values and to the second plurality of oscillation amplitude values, and wherein said controller is configured and estimate a blood pressure parameter based on the curve.

18. The system of claim 17, wherein the transducer is further configured to acquire the first plurality of oscillation amplitude values while the cuff is inflated in a stepwise manner.

19. The system of claim 17, wherein the transducer is further configured to acquire the second plurality of oscillation amplitude values while the cuff is deflated in a stepwise manner.

20. The system of claim 17, further comprising a display for displaying the blood pressure parameter.
